(19) United States
(12) Patent Application Publication
(10) Pub. No.: US 2005/0171432 A1
(43) Pub. Date: Aug. 4, 2005

(54) Detecting, assessing, and diagnosing sleep apnea

(76) Inventors: Kevin E. Crutchfield, Potomac, MD (US); B. Robert Mozayeni, Rockville, MD (US); Robert Beckman, Arlington, VA (US); Luke Beckman, Arlington, VA (US)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 10/948,218

(22) Filed: Sep. 24, 2004

Related U.S. Application Data
(63) Continuation of application No. 10/352,245, filed on Jan. 28, 2003, now abandoned.
(60) Provisional application No. 60/351,411, filed on Jan. 28, 2002.

(51) Int. Cl. A61B 8/02; A61B 8/06; A61B 8/12

(52) U.S. Cl. 600/454

(57) ABSTRACT

The present invention comprises methods for detecting, assessing, diagnosing, and pre-diagnosing sleep apnea, and for assessing the efficacy of a treatment for sleep apnea. Methods for the detection, assessment, diagnosis and pre-diagnosis (screening) of sleep apnea and the assessment of a treatment for sleep apnea according to the present invention may be performed in the absence of a sleep study. The patients subject to these methods may remain awake during their performance. The invention may be applied to other vascular conditions besides sleep apnea, wherein the sleep apnea methods described herein are example methods for the application of the present invention to the detection, assessment, diagnosis and pre-diagnosis (screening) of other vascular conditions.

This application claims priority under 35 U.S.C. § 119(e) to U.S. Provisional Patent Application No. 60/351,41 1, filed Jan. 28, 2002 and incorporated herein by reference.

### FIELD OF THE INVENTION

The invention relates to a system and method for assessing, diagnosing, and pre-diagnosing sleep apnea and assessing treatment of sleep apnea Specifically, the invention relates to a system and method for identifying critical variables, through a Dynamic Vascular Assessment (DVA) of vascular Doppler data including transcranial Doppler (TCD) data, which distinguish patients suffering from sleep apnea and the normal population.

### BACKGROUND OF THE INVENTION

Sleep apnea is a breathing disorder characterized by brief interruptions of breathing during sleep. Sleep apnea is usually caused by blockage in the lower portion of the throat, or by lack of impulse from the brain to control air passage in the respiratory system. Sleep apnea is often misdiagnosed as heart and lung problems.

Sleep apnea has many symptoms, some of which are: loud snoring, frequent night awakening, waking unrested, recent weight gain, limited attention, memory loss, headache, lethargy, or personality changes. The blood related symptoms are hallucinations, decreased consciousness, confusion, and high blood pressure.

Sleep apnea is found in all age groups and both sexes, but is more common in men and possibly young African Americans. It has been estimated that as many as 18 million Americans have sleep apnea, but many other people have been misdiagnosed, or not diagnosed at all. People most likely to have or develop sleep apnea include those who snore loudly, are overweight, have high blood pressure, or have some physical abnormality in the nose, throat, or other part of the upper airway.

There are three principle forms of sleep apnea that are currently been recognized. Obstructive sleep apnea occurs when air cannot flow into or out of the person's nose or mouth although efforts to breathe continue. This occurs when the throat and air canal are blocked, usually due to soft tissue in the rear of the throat collapsing during sleep. Central sleep apnea occurs when the brain fails to send the appropriate signals to the breathing muscles to initiate respirations. This form of seep apnea results from a lack of impulses from the brain to the respiratory system. Central sleep apnea may also be the result of diseased nerve pathways such that impulses never make it to the respiratory system. Mixed sleep apnea is a combination of both obstructive and central sleep apnea. During all of these types of sleep disorders, the brain arouses sleep apnea victims from sleep in order to resume breathing, so sleep is short and very interrupted.

When someone has a sleep apnea condition, the carbon dioxide level in his/her body rises rapidly and the oxygen level drops dramatically. The blood pH level also drops as a result of the buildup of hydrogen ions and an increased production of carbonic acid. The person will cease breathing for several seconds at a time, for up to ½ minute, and then wake up breathing hard and gasping for oxygen. The blood vessels in the body may vasodilate to increase the blood flow and move oxygen throughout the body to sustain brain functions. Vasodilation refers to the increase in the internal diameter of a blood vessel that results from relaxation of smooth muscle within the wall of the vessel. Vasodilation results in an increase in blood flow, but a decrease in systemic vascular resistance. This phenomenon puts increased stress on the cardiovascular system and may lead to an increased chance of stroke. When a person is young, blood flow is extensive and maximal vasodilation is possible. As someone gets older, the blood vessels shrink and may even constrict circulation. Thus, older people are more prone to stroke.

Conventionally, sleep apnea has been difficult to assess and diagnose accurately. There is, therefore, a need for a system and method for providing a reliable assessment of sleep apnea.

Transcranial Doppler (TCD) ultrasound has proven to be a safe, reliable, and relatively inexpensive technology for measuring cerebrovascular blood velocities. With TCD, pulses of ultrasound, at frequencies around 2 MHz, are directed using a handheld transducer towards the vascular formations in the base of the skull. The frequency shift, the Doppler effect, in the reflected sound indicates the velocity of the reflecting matter.

The Doppler effect is a change in the frequency of a wave, resulting from motion of the wave source or receiver or in the case of a reflected wave, motion of the reflector. In medicine, Doppler ultrasound is used to detect and measure blood flow, and the major reflector is the red blood cell.

Images can also be reconstructed (much like the sonography used in the evaluation of fetuses) from the time dependent intensity of the reflected sound such that vascular lesions can be visualized. Velocities from the cerebral arteries, the internal carotids, the basilar and the vertebral arteries can be sampled by altering transducer location, angle and the instrument's depth setting.

### SUMMARY OF THE INVENTION

The present invention comprises methods for detecting, assessing, diagnosing, and pre-diagnosing sleep apnea, and for assessing a treatment for sleep apnea. Methods for the detection, assessment, diagnosis and pre-diagnosis of sleep apnea and the assessment of a treatment for sleep apnea according to the present invention may be performed in the absence of a sleep-study. The patients subject to these methods may remain awake during the process. The invention may be applied to other vascular conditions besides sleep apnea, wherein the sleep apnea methods described herein are example methods for the application of the present invention to the detection, assessment, diagnosis and pre-diagnosis of other vascular conditions.

The invention is described herein with a case study with statistical results showing that vasodilation and diminished Systolic Acceleration in the blood in the left hemisphere of the brain stand-out as critical variables indicating sleep apnea. The present invention further shows that with a vessel by vessel examination, a significant difference exists between vascular data from patients having sleep apnea and a reference population in particular vessels of the brain, notably in the LC1, LM1, ROA and BA vessels, among others. Sleep apnea samples are shown to be significantly different from the reference population. The present invention utilizes this showing and provides systems and methods of pre-diagnosing sleep apnea before seeing symptoms.

The present invention further addresses four issues: (1) carbon dioxide level increase or pH decrease; (2) the effect of oxygen deprivation on sleep disorder patients; (3) the key variables regarding sleep apnea; and (4) the role of vascular Doppler data in diagnosing sleep apnea.

### BRIEF DESCRIPTION OF THE DRAWING

FIG. 1 shows an example plot of data obtained and examined according to one embodiment of the present invention.

FIG. 2 shows an example plot of geometric means of vessel segments according to one embodiment of the present invention.

FIG. 3 shows a second example plot of geometric means of vessel segments according to one embodiment of the present invention.

### DETAILED DESCRIPTION OF THE INVENTION

According to the present invention, data can be gathered for the detection, assessing, and diagnosing of sleep apnea by using a noninvasive ultrasound probe and transcranial Doppler (TCD) to capture sound waves of blood flow through the brain on a computer screen. The noninvasive probe can be applied to patients lying on a table after a bit of gel is applied to each point on the subjects at which the probe is to be applied. Those points on each subject may include the back of the neck, the eye sockets, and both temples. Alternatively, a phased-array patch system, like that disclosed in U.S. Provisional Patent Application entitled “Ultrasound Array Device” filed Jan. 10, 2003 by Mozayeni et al. to collect vascular Doppler data.

The probe or patch may then be used at these points to transmit and capture Doppler sound waves. The Doppler sound waves are indicators of the state of each vessel in each patient being studied. These Doppler sound waves may then be visualized on a computer monitor and the data they provide may be analyzed to determine a pulsatility index, a systolic acceleration, and a mean flow velocity for each vessel of each patient. The pulsatility index is a reflection of vascular impedance and small vessel resistance to pulsatile flow. During blood flow, systolic pressure is exerted on the walls of the arteries during the contraction phase of the heart, indicating the maximum arterial pressure during contraction of the left ventricle of the heart. Systolic acceleration is a measure of vessel wall force and the recovery of the walls of a vessel. The mean flow velocity is the average flow of blood transfer through a vessel segment. These data values may be extracted from the Doppler sound waves using Dynamic Vascular Assessment (DVA) and assessments of the data may be made according to the systems and methods described in U.S. Provisional Patent Application Ser. Nos. 60/236,663, 60/236,876, 60/236,661, 60/236,662, and 60/236,875, each filed Sep. 29, 2000; U.S. Provisional Patent Application Ser. Nos. 60/263,221 and 60/263,165, each filed Jan. 23, 2001; and U.S. Non-provisional Patent Application Ser. Nos. 09/966,368, 09/966,359, 09/966,367, 09/966,366, 09/966,360, and 09/682,644, each filed Oct. 1, 2001. Each of these patent applications are incorporated herein by reference in its entirety. Certain of the above patent applications describe systems that collect data and perform DVA and other operations on the data. Certain of these applications also describe services that a provider may render to users of the data or users of DVA output. It is contemplated that these systems and services may be used with the present invention such that the same systems and service providers may collect data and perform the operations described below with respect to sleep apnea and other vascular diseases.

Other data from the patients utilized by the present invention may also be gathered from a sleep center. Sleep studies can be performed on a patient overnight to observe and to measure behavior and cardiopulmonary performance while the patient tries to sleep. The patient may spend the night under observation and wear sensors and detectors which monitor him/her through the night. A sleep study may begin with a detailed sleep and wakefulness history and a neurological examination. A patient may then be asked to monitor his/her own sleep and nap schedules by keeping a diary. This may be followed, in some cases, by an overnight sleep study (polysomnogram) to observe and record nighttime sleep. Daytime wakefulness may be evaluated with a multiple sleep latency test; and a reproducible, scientific measure of sleepiness. With this information, a definitive diagnosis may be reached and an appropriate treatment plan developed.

According to particular embodiments of the present invention, the measures from such a sleep study may be combined with DVA-analyzed TCD data to form a composite record for each patient. Such a combination of multiple DVA variables and sleep apnea study data allows this invention to provide a powerful noninvasive technology to study sleep apnea and other disorders of the cerebrovascular system.

Applicants utilized the systems and methods described herein and in the thirteen U.S. patent applications listed above to perform a case example study on sleep apnea patients. This case example involved the applicants accessing, collecting, and correlating patient data and then performing statistical analysis on the data. Applicants gathered 24 patient records with available DVA and sleep study data. The 24 patients in the study were selected because they had pre-existing sleep apnea complaints and had been studied with DVA. A list of current patients with available DVA data and completed sleep studies was compiled. Patient information was coded into Microsoft Excel spreadsheets. Patient data were also loaded into Microsoft Excel spreadsheets. Coded patient records were statistically analyzed and correlated with records from a general population of about 877 people. One spreadsheet was utilized to enter DVA information. This information included data comprising 17 cranial blood vessel measurements for each patient from both the left and right hemispheres of the brain. The vessels studied included the: left and right Anterior Cerebral Arteries (LA1 and RA1); left and right Terminal Carotid Arteries (LC1 and RC1); left and right Carotid Siphon Arteries (LC4 and RC4); left and right Middle Cerebral Arteries (LM1 and RM1); left and right Ophthalmic Arteries (LOA and ROA); left and right Posterior Cerebral Arteries Toward Probe (LP1 and RP1); left and right Posterior Cerebral Arteries Away From Probe (LP2 and RP2); left and right Vertebral Arteries (LVA and RVA); and the Basilar Artery (BA). For each of the 17 vessels, two points were measured and for the two sets of two coordinates, three parameters were calculated: pulsatility index, systolic acceleration, and mean flow velocity. For the sleep studies, three different measurements were recorded comprising the total number of events hypopneas/accelerated breathing; wherein hypopnea represents a reduction in air flow or respiratory effort during sleep), lowest desaturation percentage (measure of oxygen saturation in respiration), and the respiratory disturbance index maximum (RDI), wherein RDI represents the frequency of abnormal respiratory events per hour of sleep.

Apnea is when breathing (airflow) stops for 10 seconds or more. Hypopnea is a partial blockage of airflow resulting in arousal and a possible drop in oxygen level. An RDI of 45 would indicate that the patient is experiencing complete or partial airflow blockage 45 times per hour).

In order to preserve patient confidentiality, patient identifiers and biographic data were masked by a program which produced a random 11-digit patient ID number. This number became the common key for the sleep apnea data and the DVA data. Applicants then analyzed the coded data statistically. Two sets of sleep apnea patient data were compared relative to the control group of approximately 877 patients or when DVA analysis of TCD data had been performed.

Applicants performed statistical analyses on these data by using, among other things, the Stats software pack-age MINITAB, both for each individual variable (pulsatility index, systolic acceleration, and mean flow velocity) and for a multivariate analysis of these variables. All vessels and all measures for both sleep studies data and DVA data were combined into a master table and individual statistical analyses were run for the above listed vessels of each patient.

P-values are often used in hypothesis tests, where one either rejects or fails to reject a null hypothesis. The p-value represents the probability of making a Type 1 error, wherein one rejects the null hypothesis when it is true. The smaller the p-value, the smaller the probability that one would be making a mistake by rejecting the null hypothesis. A cut-off value is often used, typically 0.05, that is one would reject the null hypothesis when the p-value is less than 0.05. For example, suppose one performs a t-test to test the null hypothesis that m equals 5, versus the alternative hypothesis that it does not equal 5. The null hypothesis that m equals 5 would be rejected if the test yields a very small (for example, less than 0.05) p-value. With respect to the statistical analyses of the present invention, a statistically significant correlation between a diminished DVA data value in a particular vessel (e.g., systolic acceleration) and sleep apnea patients would be assumed if the P-value for that data value for that vessel was lower than 0.05.

The present invention may utilize any appropriate statistical analysis to show such a significant difference. Two such analyses are the parametric T-test and the non-parametric Mann-Whitney test. The statistical formalism used to ascertain sensitivity and specificity involves establishing receiver-operator curves and discrimination thresholds.

According to the case example described herein, a statistical analysis including a Parametric T-test showed that the following vessels exhibited a significant difference between the data in the sleep apnea population and the data in the reference population: LC1, LM1, ROA, and BA. A statistical analysis including a Non-parametric Mann-Whitney test showed that the following vessels also show a significant difference: LA1, LC1, LM1, RC4, ROA, RVA, and BA. Table 1 shows the Mann-Whitney and T-test p-values that were calculated for each of 17 vessels that were analyzed in the case example according to the present invention. The following vessels, then, show a significant difference in both tests: LC1, LM1, ROA and BA.

TABLE 1
Parametric and Non-parametric Test Results

| | Equality of | |
|---|---|---|
| | Samples | |
| | Mann-Whitney | T-test |
| Vessel | p-values | p-values |
| LA1 | 0.0081 | 0.063 |
| LC1 | 0.0053 | 0.026 |
| LC4 | 0.2586 | 0.395 |
| LM1 | 0.0101 | 0.022 |
| LOA | 0.7375 | 0.820 |
| LP1 | 0.2335 | 0.378 |
| LP2 | 0.2578 | 0.433 |
| LVA | 0.3910 | 0.389 |
| RA1 | 0.1229 | 0.064 |
| RC1 | 0.9148 | 0.667 |
| RC4 | 0.0005 | 0.085 |
| RM1 | 0.0792 | 0.193 |
| ROA | 0.0027 | 0.006 |
| RP1 | 0.0940 | 0.074 |
| RP2 | 0.0875 | 0.258 |
| RVA | 0.0259 | 0.059 |
| BA | 0 | 0 |

Another statistical analysis that may be performed as part of the present invention is a paired test. As part of a paired test, a centroid analysis may be run to compare the mean centers for the reference population to the mean centers for the sleep apnea test patients. Centroid analysis regards the centroids/centroid of a cluster. The centroid is the middle of a cluster, comprising a vector containing one number for each variable, where each number is the mean of a variable for the observations in that cluster. The centroid can be used as a measure of cluster location. For a-given cluster, the average distance from the centroid is the average of the distances between observations and the cluster centroid. The maximum distance from the centroid is the maximum of these distances.

In the case example, the paired test showed that the sleep apnea patient data exhibited a significant positive increase in the mean in distance from the centroid as compared to the reference population data. The data cluster for each of the reference group and the sleep apnea group were visualized as a three-dimensional nomogram having an axis for each data value (e.g., velocity, systolic acceleration, and pulsatility index). A two-dimensional figure may also be plotted having axis for only two data values (e.g., velocity and systolic acceleration). For the two clusters, the average distance from the centroid of each group was a measurable distance and was statistically significant.

Table 2 shows mean distances from the centroid that were calculated for each of 17 vessels that were analyzed with the paired test in the case example according to the present invention Table 2 includes mean distances for the sleep apnea trial group and the reference group as well as the difference in those distances for each of the 17 vessels.

TABLE 2
Paired Test Results

| | Mean in distance from centroid | | |
|---|---|---|---|
| Vessel | Trial | Reference | Difference |
| LA1 | 0.683071 | 0.208302 | 0.47477 |
| LC1 | 0.836004 | 0.188917 | 0.64709 |
| LC4 | 0.410453 | 0.112322 | 0.29813 |
| LM1 | 0.616470 | 0.204837 | 0.41163 |
| LOA | 0.188406 | 0.144308 | 0.04410 |
| LP1 | 0.379289 | 0.223884 | 0.15541 |
| LP2 | 0.430028 | 0.227113 | 0.20291 |
| LVA | 0.276540 | 0.098173 | 0.17837 |
| RA1 | 0.662840 | 0.215663 | 0.44718 |
| RC1 | 0.438529 | 0.183774 | 0.25476 |
| RC4 | 0.656524 | 0.275515 | 0.38101 |
| RM1 | 0.640544 | 0.219408 | 0.42114 |
| ROA | 0.679476 | 0.130558 | 0.54892 |
| RP1 | 0.582673 | 0.181960 | 0.40071 |
| RP2 | 0.458258 | 0.169114 | 0.28914 |
| RVA | 0.570393 | 0.188537 | 0.38186 |
| BA | −0.699220 | 0.233560 | −1.16533 |

The sleep apnea group showed a distinctive shift to the left from the reference centroid. This shift shows that the critical variables for sleep apnea patients can be isolated. The shift left corresponded to a decreased systolic acceleration value, which indicates that vasodilation and diminished systolic acceleration in the left hemisphere of the brain of sleep apnea patients stands out as significant when compared with a reference population. Sleep apnea patients thus present a lower systolic acceleration value as compared to the normal population.

Other variables regarding the cerebrovascular system also exhibit a measurable correlation between subjects with sleep apnea and the normal population. Of the 54 measurements taken from a DVA test, the vessels shown to have a significant correlation with sleep apnea included the left anterior cerebral artery (LA1), left terminal carotid artery (LC1), left middle cerebral artery (LM1), right carotid siphon artery (RCA), right ophthalmic artery (ROA), right vertebral artery (RVA), and basilar artery (BA).

The present invention thus comprises a methods for assessing, diagnosing, or pre-diagnosing sleep apnea in patients before seeing symptoms or before actual diagnosis via a sleep study. An automated system may also be used to perform some or all of the steps of such methods.

For example, according to one embodiment of the present invention, DVA data may be collected for any one or more of the LC1, LM1, LA1, RCA, RVA, and BA vessels from a patient. Then, systolic acceleration, mean flow velocity, and/or pulsatility index values may be calculated from the DVA data. These data may be visualized as compared to cluster from a set of corresponding data values for the same vessels in a reference population. A centroid analysis may then be performed and if the patient's data reflect a significant positive increase in the mean distance from the centroid of the reference data, then the patient may be diagnosed as having sleep apnea.

According to another embodiment of the present invention, a treatment for sleep apnea may be assessed based on the above described analysis methods. For example, DVA data may be collected prior to the administration of a treatment for any one or more of the LC1, LM1, LA1, RCA, RVA, and BA vessels from a patient believed to have sleep apnea Then, systolic acceleration, mean flow velocity, and/or pulsatility index values may be calculated from the DVA data. A treatment may then be administered to the patient. A second set of DVA data may be collected after the administration for any one or more of the same vessels measured prior to the treatment. A cluster derived from the values obtained from the pre-treatment data may be visualized as compared to cluster derived from the values obtained after the administration of the treatment. A centroid analysis may then be performed and if the patient's data after the treatment reflect a shift towards the normal range (e.g., if the systolic acceleration shifts right, or increases) and away from the cluster represented by the patient's data before the treatment, then the treatment may be assessed as having a reductive effect on the patient's sleep apnea. If, however, there is no significant shift between the centroid of the post-treatment data from the pre-treatment data; then the treatment may be assessed as having little therapeutic effect on the patient's sleep apnea The post-treatment data may be collected at any point after the administration of a treatment. In some embodiments of the present invention, more than one or ongoing assessments of a treatment may be made, wherein more than one post-treatment data set is collected and compared against each other set of post-treatment and/or pre-treatment data. Alternatively, any set of post-treatment data may be compared to reference population data instead of or in addition to pre-treatment data collected from the same patient.

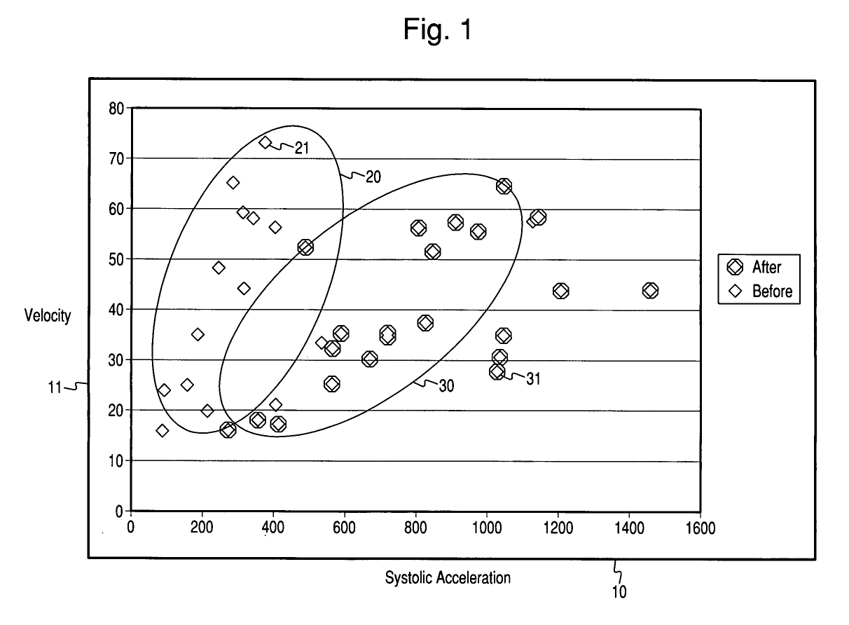

FIG. 1 shows pre-treatment points 21 and post-treatment points 31 plotted as systolic acceleration values on systolic acceleration axis 10 and velocity values on velocity axis 11, where the systolic acceleration and velocity values were derived from data collected from a patient having sleep apnea and relate to certain cerebral vessels. Pre-treatment points 21 are shown forming pre-treatment centroid 20 and post-treatment points 31 are shown forming post-treatment centroid 30. Based on the example shown in FIG. 1, it may be determined that the treatment administered to the patient had a reductive effect on the patient's sleep apnea as post-treatment centroid 30 shows a shift to the right along systolic acceleration axis 10 (i.e., there is a greater systolic acceleration) as compared to pre-treatment centroid 20.

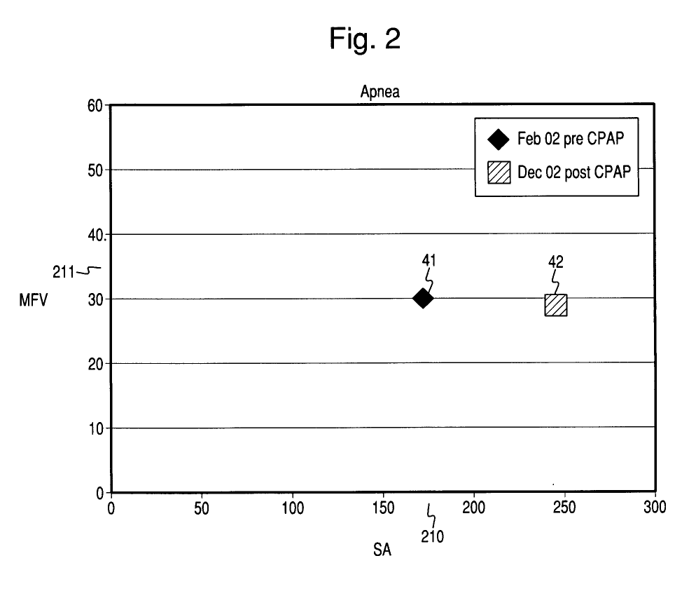

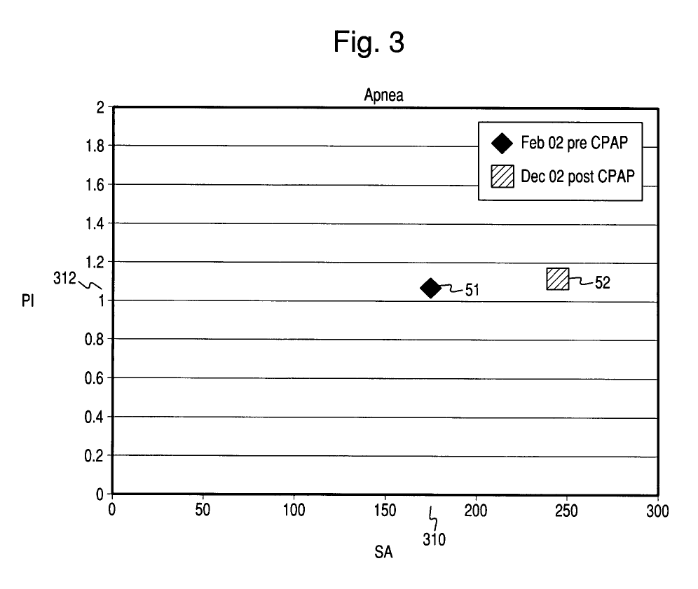

FIGS. 2 and 3 show the geometric means of all vessel segments of the same patient whose data is represented in FIG. 1. FIG. 2 shows the geometric means at a time pre-CPAP 41 and at a time post-CPAP 42 as plotted along asystolic acceleration axis 210 and a velocity axis 211. FIG. 2 shows a shift in the geometric means to the right along systolic acceleration axis 210 from pre to post-CPAP.

FIG. 3 shows the geometric means at a time pre-CPAP 51 and at a time post-CPAP 52 as plotted along a systolic acceleration axis 310 and a pulsatility axis 312. FIG. 3 also shows a shift in the geometric means to the right along systolic acceleration axis 310 from pre to post-CPAP.

An analysis of such geometric means may also or may instead be used as a statistical analysis in accordance with the present invention

It is a key feature of the present invention that the above methods of diagnosis of sleep apnea and methods for the assessment of treatment for sleep apnea may be performed while a patient is awake. A sleep study may be used to supplement the data used in these methods according to the present invention, but one is not required for the performance of these methods.

DVA of TCD data may be used according to the present invention to isolate previously unknown evidence that sleep apnea has cerebrovascular effects that may be used for screening or may be diagnostically used to verify whether sleep apnea patients have global dilation throughout the body, and possibly may experience dilated blood vessels during normal hours of activity.

The use of DVA in the above way regarding sleep apnea is also helpful for general vascular science. DVA according to the present invention may identify what vessels are contributing to this diminished oxygen capacity and how does the brain compensate for the diminished capacity. Other diseases may also be evaluated in a like manner using the technology of the present invention. These diseases include, but are not limited to stroke and general neurological dysfunctions.

1. A method of diagnosing or assessing sleep apnea comprising the steps of:
referencing one or more blood flow values for each of one or more patient vessels;
referencing one or more blood flow values for each of one or more reference population vessels, wherein the one or more reference population vessels are corresponding vessels to the one or more patient vessels;
statistically analyzing the difference in the one or more blood flow values for each one or more patient vessels from the one or more blood flow values for each one of the corresponding one or more reference population vessels; and
determining whether sleep apnea exists or to what extent sleep apnea exists in a patient based on said difference.

2. The method of diagnosing or assessing sleep apnea according to claim 1, wherein the blood flow values referenced for both the one or more patient vessels and the one or more reference population vessels comprise one or more of: systolic acceleration, mean flow velocity, and pulsatility index.

3. The method of diagnosing or assessing sleep apnea according to claim 2, wherein the blood flow values referenced for both the one or more patient vessels and the one or more reference population vessels comprise systolic acceleration, and wherein said difference comprises a systolic acceleration for each one or more patient vessels that is less than a systolic acceleration for each one of the corresponding one or more reference population vessels.

4. The method of diagnosing or assessing sleep apnea according to claim 2, wherein the blood flow values referenced for both the one or more patient vessels and the one or more reference population vessels consist of: systolic acceleration and mean flow velocity.

5. The method of diagnosing or assessing sleep apnea according to claim 2, wherein the blood flow values referenced for both the one or more patient vessels and the one or more reference population vessels consist of: systolic acceleration, mean flow velocity, and pulsatility index.

6. The method of diagnosing or assessing sleep apnea according to claim 1, wherein the one or more patient vessels and the corresponding one or more reference population vessels are located in the left hemisphere of the patient's brain.

7. The method of diagnosing or assessing sleep apnea according to claim 1, wherein the one or more patient vessels and the corresponding one or more reference population vessels comprise one or more of: left anterior cerebral artery, left terminal carotid artery, left middle cerebral artery, right carotid siphon artery, right ophthalmic artery, right vertebral artery, and basilar artery.

8. The method of diagnosing or assessing sleep apnea according to claim 7, wherein the one or more patient vessels and the corresponding one or more reference population vessels comprise one or more of: left terminal carotid artery, left middle cerebral artery, right ophthalmic artery, and basilar artery.

9. The method of diagnosing or assessing sleep apnea according to claim 7, wherein the one or more patient vessels and the corresponding one or more reference population vessels consist of left terminal carotid artery, left middle cerebral artery, right ophthalmic artery, and basilar artery.

10. The method of diagnosing or assessing sleep apnea according to claim 1 further comprising the step of:
referencing a sleep study performed on the patient, and wherein the step of determining whether sleep apnea exists or to what extent sleep apnea exists in a patient is based on said difference and on the sleep study.

11. The method of diagnosing or assessing sleep apnea according to claim 1, wherein the step of statistically analyzing the difference in blood flow values comprises one or more of a centroid analysis, a parametric test, or a non-parametric test.

12. The method of diagnosing or assessing sleep apnea according to claim 1 further comprising the step of:
calculating the one or more blood flow values for the one or more patient vessels from patient blood flow data

13. The method of diagnosing or assessing sleep apnea according to claim 12 further comprising the step of:
collecting the patient blood flow data from a patient.

14. The method of diagnosing or assessing sleep apnea according to claim 13 further comprising the step of:
collecting the patient blood flow data using vascular Doppler ultrasound.

15. The method of diagnosing or assessing sleep apnea according to claim 12 further comprising the step of:
calculating the one or more blood flow values for the one or more reference population vessels from reference population blood flow data

16. The method of diagnosing or assessing sleep apnea according to claim 15 further comprising the step of:
collecting the reference population blood flow data from one or more subjects from a general population.

17. The method of diagnosing or assessing sleep apnea according to claim 16 further comprising the step of:
collecting the reference population blood flow using vascular Doppler ultrasound.

18. The method of diagnosing or assessing of diagnosing sleep apnea according to claim 1, wherein the blood flow values referenced for both the one or more patient vessels and the one or more reference population vessels are calculated from blood flow data, and wherein the blood flow data is collected using a means for vascular imaging.

19. The method of diagnosing or assessing of diagnosing sleep apnea according to claim 18, wherein the means for vascular imaging comprises a vascular Doppler ultrasound probe.

20. The method of diagnosing or assessing of diagnosing sleep apnea according to claim 1, wherein the method is performed while the patient is awake.

21. A method of diagnosing or assessing a vascular disease comprising the steps of:
referencing one or more blood flow values for each of one or more patient vessels;
referencing one or more blood flow values for each of one or more reference population vessels, wherein the one or more reference population vessels are corresponding vessels to the one or more patient vessels;
statistically analyzing the difference in the one or more blood flow values for each one or more patient vessels from the one or more blood flow values for each one of the corresponding one or more reference population vessels; and
determining whether a vascular disease exists or to what extent sleep apnea exists in a patient based on said difference;
wherein the blood flow values referenced for both the one or more patient vessels and the one or more reference population vessels are calculated from blood flow data, and wherein the blood flow data is collected using vascular Doppler ultrasound.

22. The method of diagnosing or assessing a vascular disease according to claim 21, wherein the blood flow values referenced for both the one or more patient vessels and the one or more reference population vessels comprise one or more of: systolic acceleration, mean flow velocity, and pulsatility index.

23. The method of diagnosing or assessing a vascular disease according to claim 22, wherein the blood flow values referenced for both the one or more patient vessels and the one or more reference population vessels consist of: systolic acceleration, mean flow velocity, and pulsatility index.

24. The method of diagnosing or assessing a vascular disease according to claim 21, wherein the step of statistically analyzing the difference in blood flow values comprises one or more of a centroid analysis, a parametric test, or a non-parametric test.

25. The method of diagnosing or assessing a vascular disease according to claim 21, wherein the vascular disease is sleep apnea.

26. The method of diagnosing or assessing a vascular disease according to claim 21, wherein the vascular disease is stroke.

27. The method of diagnosing or assessing of diagnosing sleep apnea according to claim 21, wherein the method is performed while the patient is awake.

28. A method of assessing the effectiveness of a treatment for sleep apnea comprising the steps of:
referencing one or more first blood flow values for each of one or more patient vessels, the first patient vessel values being calculated before the administration of a treatment;
referencing one or more second blood flow values for each of one or more patient vessels, the second patient vessel values being calculated after the administration of a treatment;
statistically analyzing the difference in the one or more first blood flow values from the one or more second blood flow values; and
determining the effectiveness of the treatment based on said difference;
wherein the one or more first blood flow values and the one or more second blood flow values are calculated from blood flow data, and wherein the blood flow data is collected using vascular Doppler ultrasound.

29. The method of assessing the effectiveness of a treatment for sleep apnea according to claim 28, wherein the blood flow values referenced both before and after the administration of the treatment comprise one or more of: systolic acceleration, mean flow velocity, and pulsatility index

30. The method of assessing the effectiveness of a treatment for sleep apnea according to claim 29, wherein the blood flow values referenced both before and after the administration of the treatment consist of systolic acceleration, mean flow velocity, and pulsatility index.

31. The method of assessing the effectiveness of a treatment for sleep apnea according to claim 28, wherein the step of statistically analyzing the difference in blood flow values comprises one or more of a centroid analysis, a parametric test, or a non-parametric test.

32. The method of assessing the effectiveness of a treatment according to claim 28, wherein the one or more patient vessels are located in the left hemisphere of the patient's brain.

33. The method of assessing the effectiveness of a treatment according to claim 28, wherein the one or more patient vessels comprise one or more of: left anterior cerebral artery, left terminal carotid artery, left middle cerebral artery, right carotid siphon artery, right ophthalmic artery, right vertebral artery, and basilar artery.

34. The method of assessing the effectiveness of a treatment according to claim 33, wherein the one or more patient vessels comprise one or more of: left terminal carotid artery, left middle cerebral artery, right ophthalmic artery, and basilar artery.

35. The method of assessing the effectiveness of a treatment according to claim 34, wherein the one or more patient vessels consist of: left terminal carotid artery, left middle cerebral artery, right ophthalmic artery, and basilar artery.

36. The method of assessing the effectiveness of a treatment according to claim 28, wherein the method is performed while the patient is awake.

37. A method of diagnosing or assessing sleep apnea in a patient comprising the steps of:
referencing one or more blood flow values of the patient;
referencing one or more blood flow values for a reference population, wherein the one or more reference population blood flow values correspond to the one or more patient blood flow values;
statistically analyzing the difference between the one or more patient blood flow values and the one or more corresponding reference population blood flow values; and
determining whether sleep apnea exists or to what extent sleep apnea exists in a patient based on said difference;
wherein the patient is awake.

38. The method of diagnosing or assessing sleep apnea according to claim 37, wherein the one or more patient blood flow values and the corresponding one or more reference population blood flow values are derived from cerebrovascular data.

39. The method of diagnosing or assessing sleep apnea according to claim 38, wherein the cerebrovascular data is vascular Doppler ultrasound data

40. The method of diagnosing or assessing sleep apnea according to claim 38, wherein the cerebrovascular data is collected from the left hemisphere of the patient's brain.

41. The method of diagnosing or assessing sleep apnea according to claim 38, wherein the cerebrovascular data is collected from one or more of left terminal carotid artery, left middle cerebral artery, right ophthalmic artery, and basilar artery.

42. The method of diagnosing or assessing sleep apnea according to claim 37, wherein the one or more patient blood flow values and the corresponding one or more reference population blood flow values comprise one or more of systolic acceleration, mean flow velocity, and pulsatility index.
